(19) United States
(12) Patent Application Publication
(10) Pub. No.: US 2019/0328408 A1
(43) Pub. Date: Oct. 31, 2019

(54) ARTHROPLASTY TOOL AND METHODS FOR USING SAME

(71) Applicant: Brad L. Penenberg, Los Angeles, CA (US)

(72) Inventor: Brad L. Penenberg, Los Angeles, CA (US)

(21) Appl. No.: 16/395,168

(22) Filed: Apr. 25, 2019

Related U.S. Application Data
(60) Provisional application No. 62/662,223, filed on Apr. 25, 2018.
(60) Provisional application No. 62/722,829, filed on Aug. 24, 2018.

(51) Int. Cl. A61B 17/17; A61B 17/16; A61F 2/46

(52) U.S. Cl. CPC A61B 17/1746; A61B 2017/00734; A61F 2/4609; A61B 17/1666

(57) ABSTRACT

An apparatus and improved methods for hip arthroplasty, including by introducing a modular rod that may remain resident in the path to the acetabulum and receive a series of tools or have segmented sections of tools to perform separate functions during the surgical procedure in which the incision length and risk of inadvertent surgical trauma are reduced.

### CROSS-REFERENCES TO RELATED APPLICATIONS

This application claims the benefit of Provisional Patent Application Ser. No. 62/662,223, filed Apr. 25, 2018, and Provisional Patent Application Ser. No. 62/722,829, filed Aug. 24, 2018, both of which are incorporated herein by this reference.

### BACKGROUND OF THE INVENTION

The present invention pertains to minimally invasive surgical techniques and tools. In particular, techniques and instruments for simpler, faster, and/or more reliable and precise operations.

In general, minimally invasive techniques for partial or total hip arthroplasty often entail making a main incision in the hip area to access the acetabulum and making a separate portal incision spaced a given distance from the main incision to access the acetabulum roughly face on, such as perpendicularly to the plane of the acetabulum. The surgeon thereby may introduce one or more instruments through the portal incision to access and prepare the acetabulum and then to place and manipulate an acetabular component. Sometimes these steps are facilitated by first placing a cannula, or other thin walled metal or plastic tube, through this portal incision for subsequent passage of instruments while minimizing contact with any of the surrounding soft tissue, blood vessels, etc.

Likewise, the femur may be prepared for receiving a femoral implant and to receive the ball that will be coupled with the acetabular component, often by introducing instruments through the main incision and/or a separate, femoral portal incision spaced a distance away from the main incision. Similarly, these steps are sometimes facilitated by first introducing such a cannula so that the instruments do not significantly brush against soft surrounding tissues.

### SUMMARY OF THE INVENTION

This invention relates to an apparatus and improved methods for hip arthroplasty, also referred to as hip replacement, and more particularly, arthroplasty in which the incision length and risk of inadvertent surgical trauma are reduced.

### BRIEF DESCRIPTION OF THE DRAWINGS

FIG. 1 shows a diagram of a modular rod in keeping with one embodiment of the present invention with a connector region and a cone tip inserted into an incision.

FIG. 2A shows a modular rod in keeping with one embodiment of the present invention with one embodiment of a guide wire.

FIG. 2B shows a modular rod in keeping with one embodiment of the present invention with one embodiment of a guide wire.

FIGS. 3A and 3B show other, potential connector regions for attaching various tools for connecting to the modular rod.

FIGS. 4A and 4B show an embodiment of a burr for connecting to the modular rod.

FIG. 5 shows a cutting tool attachment in keeping with one embodiment of the present invention.

FIG. 6 shows a plurality of reamers for attaching to the modular rod.

FIG. 7 shows an attachment for positioning the acetabular component.

FIG. 8 shows a cup placement device for inserting a new acetabular component.

FIG. 9A shows various potential connector types that may be implemented into the modular rod.

FIG. 9B shows a perspective view of an impactor showing the connector region and introducer cone cutout for attaching to the modular rod.

FIG. 10 shows a stabilizing disk for attaching to the modular rod.

FIG. 11 shows another embodiment of the modular rod in keeping with the present invention and various potential connector types that may be implemented into the modular rod.

FIG. 12 shows another embodiment of the modular rod in keeping with the present invention.

### DESCRIPTION OF THE PREFERRED EMBODIMENTS

The detailed description set forth below in connection with the appended drawings is intended as a description of presently-preferred embodiments of the invention and is not intended to represent the only forms in which the present invention may be constructed and/or utilized. The description sets forth the functions and the sequence of steps for constructing and operating the invention in connection with the illustrated embodiments. However, it is to be understood that the same or equivalent functions and sequences may be accomplished by different embodiments that are also intended to be encompassed within the spirit and scope of the invention.

One advantage of an embodiment herein is the ability to perform surgeries as described in the background without the use of a cannula. Before surgery, the surgeon determines the location of a portal incision 101 using an X-ray or fluoroscopy and places a metal marker at approximately the level of the lesser trochanter (for the posterior approach method) or the base of the greater trochanter (for the anterior approach method) as estimated by palpating the bony landmarks. The portal incision 101 location may be at approximately the level of the lesser trochanter as identified on a radiograph of the pelvis. This will verify the approximate portal incision 101 location. When using a posterior approach, this portal incision 101 will be posterior to the femur. When using an anterior approach, the portal incision 101 will be located anterior to the femur and greater trochanter.

In some embodiments of the present invention, for sighting the portal incision 101 location, bony landmarks may be utilized. This technique may be utilized in hip and knee arthroscopy traction pin placement in trauma cases. For a superior or minimally invasive total hip arthroplasty (“THA”), the base of the greater trochanter may be palpated, which makes the process easier due to the trochanter being readily identified through the main incision 105. With the limb in a neutral or slight internal rotation, there may be approximately 20 to 30 degrees of flexion and a slight abduction. After palpating the base of the greater trochanter, the proximal distal entry level may be identified approximately one inch, or two fingerbreadths, distally. The posterior edge of the femur may be identified and a final location may be chosen approximately a centimeter posterior to the edge of the femur at the portal incision location previously determined. For example, a one centimeter incision may be made and a multi-function modular rod 110 as described below may be placed and directed toward a readily visualized center of the acetabulum 115.

In some embodiments, the surgeon may use a sensor-directed portal placement guide system. The sensor-directed portal placement guide system would entail a trocar, guide rod, guide wire 120 or the like, along with one or more remote positioning sensors and transmitters. The remote positioning sensors and transmitters detect and send information regarding their position(s) and orientation(s) relative to adjacent anatomical landmarks, such as a bone surface, acetabulum, femoral neck of head, or other significant anatomical structure. The guide wire 120 may have its own sensors and/or transmitter for establishing the position and orientation of the guide wire 120, and the two (the remote sensors and the sensors on the guide) may work in conjunction with one another and/or with a computer processor to confirm a portal incision 101 and direction that is as sparing as possible on any nearby critical organs, arteries, veins, or soft tissue.

The sensor information could also be combined with imaging information, such as intermittent or continuous x-ray imaging that can provide the position and orientation of landmarks in three dimensions, such as the circular face of the acetabulum 115, structures on the greater trochanter or femoral neck, or the like. This three-dimensional data can be used along with a similar three-dimensional imaging or sensing of the guide wire 120, during the surgery, in order to reach the target, such as the center of the acetabulum 115, with a great degree of precision and accuracy, for the skilled surgeon and for a newer surgeon as well. Furthermore, some or most of these steps could be performed by automation or robotically guided surgery.

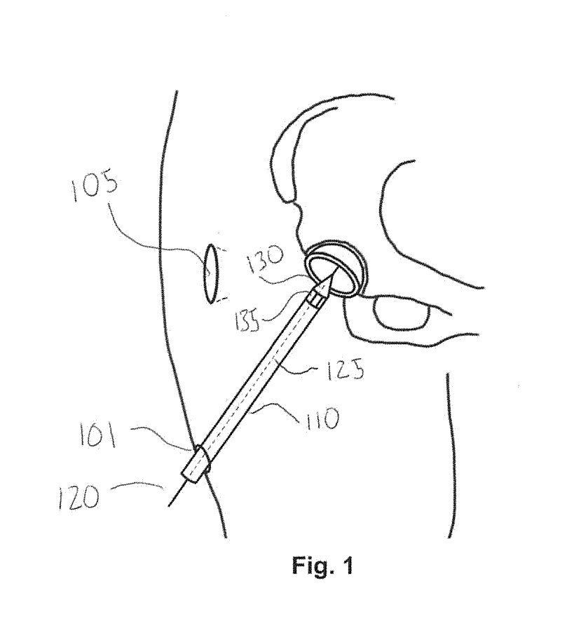

In some embodiments, a guide wire 120 may be inserted through the portal incision 101 to the acetabulum. The guide wire 120, such as the one illustrate in FIG. 1, generally has a thin diameter, for example, less than 5 mm in diameter, but is generally sufficiently stiff to be passed through the tissue without significant bending. The path of the guide wire 120 can be determined using radiography techniques to avoid or minimize tissue damage. Once the guide wire 120 is in place, the surgeon may use the guide wire 120 to guide a multi-function modular rod member 110 towards the acetabulum 115 by feel, imaging, or the like.

Cannulas can present their own risks to surrounding soft tissues, particularly at the tip where the thin walled tube represents a relatively sharp edge close to or within the hip joint. The cannula, even after it is in place, starts to move in and out of the soft tissues each time a surgeon introduces and removes the cannula. One embodiment of the present invention does not have sharp edges therefore, avoids using a cannula or uses a modified cannula to minimize sharp edges. In such cases, the modular rod does not have sharp edges or at least minimizes sharp edges, and, as a result, trauma to nearby tissues is reduced, as is any bleeding, post-surgical pain, and post-surgical recovery time. Even small movements of the cannula can damage nearby tissues. Muscle tissue, tendons and blood vessels all can be sliced by the cannula edges leading to trauma, excess bleeding, post-operative pain, and slow recovery following the surgery.

Sometimes, in fact, the cannula has to be redirected over much of the length of the incision channel. With each passage through the incision channel, especially in the downward direction, into the hip cavity, the sharp edges of the cannula can make new cuts in the nearby tissues causing more trauma to the tissues and creating more bleeding. The cannula, if reinserted into the channel, would therefore create additional trauma as it would typically have to cut anew through the soft tissues. As a result, the present invention reduces trauma caused by sharp edges by using a device with generally smooth sides, referred to herein as a modular rod 110, which can be guided to the surgical site with the use of the guide wire. Surgical tools having the desired characteristics can then be attached to the modular rod 110 to perform the desired function at the surgical site. Without removing the modular rod 110 from the surgical site completely, the current surgical tool attached to the modular rod 110 can be removed and exchanged for another surgical tool to perform a different function while minimizing movement of the surgical rod 110 to effectively minimize potential trauma to the tissue.

Modular Rod

The modular rod 110 can be an elongated shaft that defines a longitudinal axis, the shaft having an access end 185 and an insertion end 180. The access end 185 provides an area for the surgeon to hold the modular rod 110 during use so the surgeon can insert, manipulate, and withdraw the modular rod 110 during use. The insertion end 180 is the end that is inserted into the subject to perform the necessary functions. The modular rod 110 can be generally solid and having a length of approximately 6 inches to approximately 12 inches, and a diameter of approximately 6 mm to approximately 12 mm, and preferably about 10 mm in diameter.

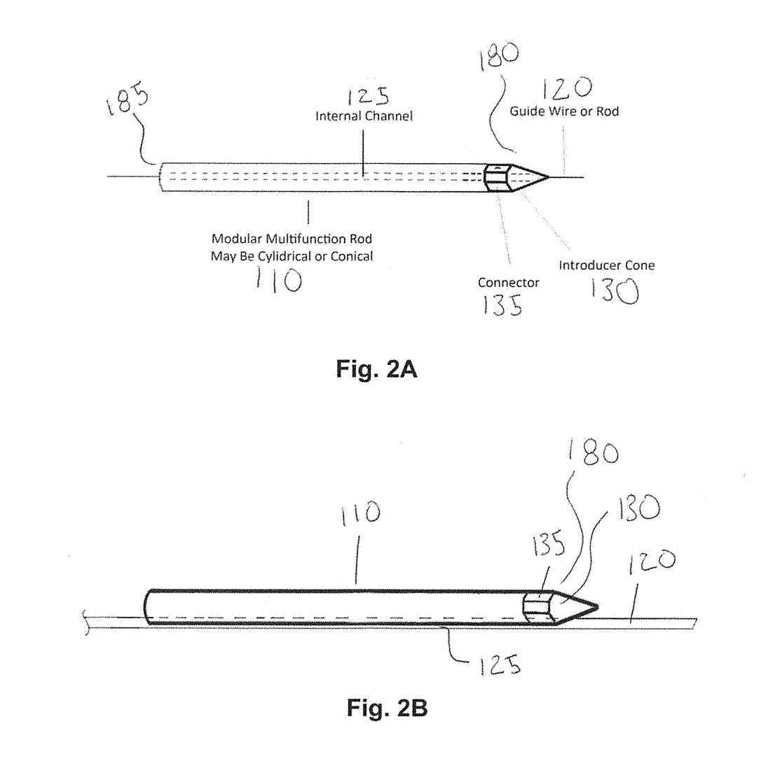

The modular rod 110 may have a channel 125 that extends the length of the modular rod 110. In some embodiments, the channel 125 may be created along the longitudinal axis of the shaft, essentially co-axial with the longitudinal axis of the modular rod 110 as shown in FIG. 2A. In some embodiments, as shown in FIG. 2B, the channel 125 for receiving a guide wire 120 can be external to the shaft or off-set from the longitudinal axis. Thus, channel 125 can be an open channel or a generally U-shaped channel in which the channel is not completely enclosed in the shaft. In some embodiments, the channel can be parallel to, but off-center from, the longitudinal axis. In some embodiments, the main body of the modular rod 110 may be oval or any other shape to accommodate the location of the channel 125.

The channel 125 allows the modular rod 110 to be guided towards the acetabulum with the assistance of the guide wire 120. With the guide wire 120 in place, whose position can be confirmed radiographically, the modular rod 110 may be positioned so as to allow the guide wire 120 to be inserted through the channel 125 so that the modular rod 110 may be inserted through the tissue along the guide wire 120. As such, the channel 125 of the modular rod 110 may be sized to be substantially similar or slightly larger than the diameter of the guide wire 110. The modular rod 110 may slide along the guide wire 120 with little resistance to prevent bending or damage to the guide wire 120.

In some embodiments, the modular rod 110 may be made from a material that is translucent so the surgeon can see the guide wire 120 through the modular rod 110. If either the guide wire 120 or the modular rod 110 is radio-opaque, placement can occur using radiographic guidance. This can include fluoroscopy or individual digital images.

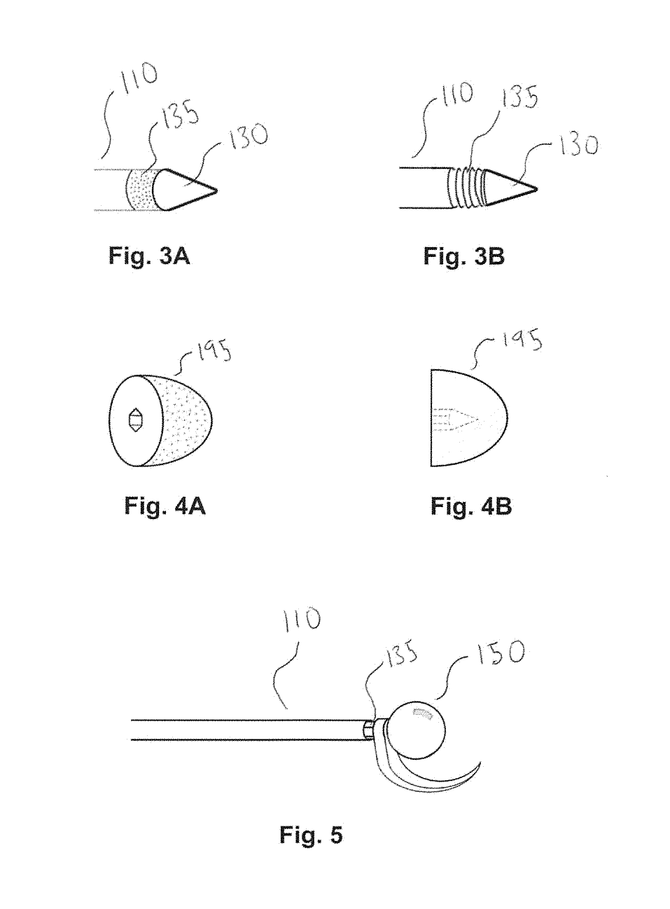

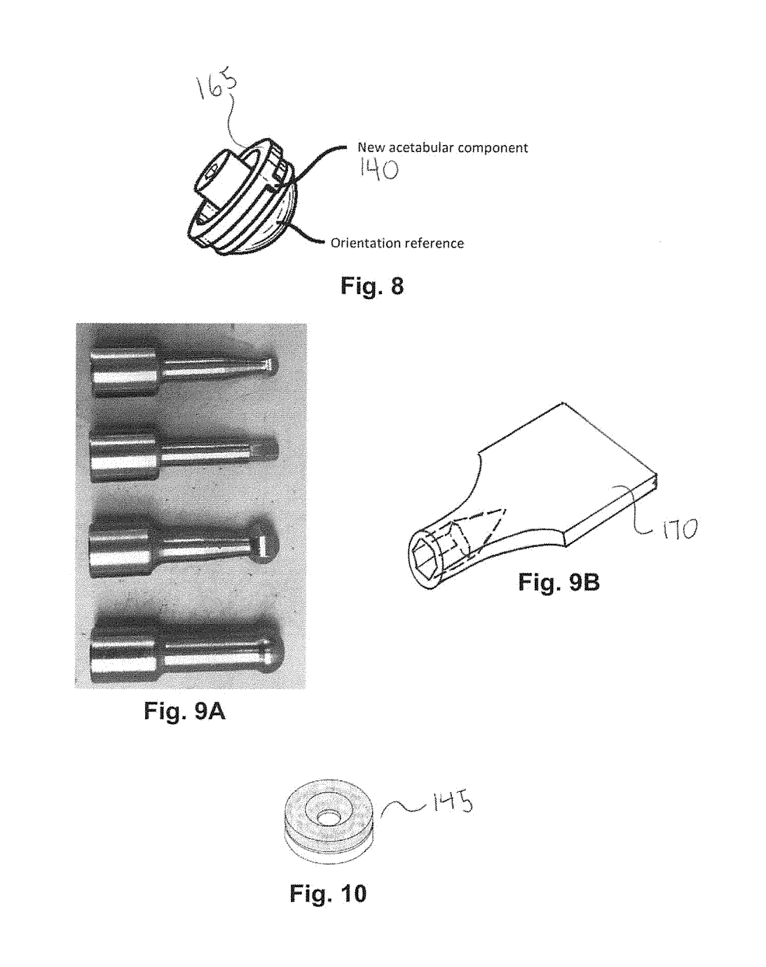

The insertion end 180 of the modular rod 110 may have one or more connector regions 135. The connector region 135 allows for the attachment of a variety of different surgical tools, such as cutting tools (see, FIGS. 3 through 10), a cup placement device, a cup driver and director, a standard or articulating screwdriver module (see, FIG. 8), a specially created acetabular component removal blade or cutter (see, FIG. 5), a burr 195 (see, FIGS. 4A and 4B), a compactor, and any other tool used to prepare the acetabulum to receive an acetabular component 140 when necessary.

To attach a surgical tool to the connector region 135, the connector region 135 may be configured with any type of fastener that would allow for easy attachment and removal of attachment tools to the connector region 135. For example, the connector region 135 may be threaded (FIG. 3A), keyed (e.g., by use of a hex key or slotted, etc., see FIGS. 2A and 2B), socketed, and/or have detents, catches, clips, magnets, hooks, chucks, clamps, resistance fit material (FIG. 3A), and the like, or any combination thereof. Any surgical tool that can attach to the connector region 135 may also include a reciprocal fastener configuration so that the connector region 135 can securely mate with the surgical tools.

The connector region 135 may be integrally formed with the insertion end 180 of the modular rod 110 or fastened to the modular rod 110. In some embodiments, the connector region 135 may be removably fastened to the modular rod 110, using any of the fasteners used to connect the connector region 135 to the surgical tool, so that the connector region 135 can be removed from the modular rod 110.

Internally, the connector region 135 may also have a channel that is continuous with the channel 125 of the modular rod 110. This will allow the guide wire 120 to pass through the channel of the connector region 135.

Introducer Cone

In some embodiments, attached to the connector end 135 may be an introducer cone 130. The introducer cone 130 may be a cone-shaped device having adequate sharpness at its tip to penetrate the tissues, but also a smooth sidewall to create a soft tissue channel with minimal damage to the tissue as the modular rod 110 is inserted through the tissue. In some embodiments, the introducer cone 130 may not have any sharp edges at the front end of the modular rod 110. For example, the introducer cone 130 may have a smooth taper. In some embodiments, the introducer cone 130 may have a torpedo or bullet-like shape.

FIG. 1 shows one embodiment of a modular rod 110 with an introducer cone 130 and a connector region 135 being guided along a guide wire 120 through a portal incision 101 to the acetabulum 115. FIGS. 2A and 2B illustrate a connector region 135 behind the introducer cone 130, and FIGS. 3A and 3B illustrate other such connector structures 135 behind the introducer cone 130.

The introducer cone 130 may be integrally formed on the front end or the insertion end 180, or securably attached to the insertion end 180. Preferably, the introducer cone 130 may be integrally formed or connected to the connection region 135 in a manner that can be removed from the connection region 135. The surgeon may thereby urge the modular rod 110 down over the guide wire 120 and into the path forged by the guide wire 120 to guide the multi-function modular rod 110 to the acetabulum. The tapered shape of the introducer cone 130 creates a larger path through the tissue than the guide wire; however, because of the smooth surface of the introducer cone 130, tissue damage is minimized.

Internally, the introducer cone 130 may also have a channel that is continuous with the channel 125 of the modular rod 110 and the connector region 135 to allow the guide wire 120 to pass through.

The surgical tools that are attached to the connector region 135 are configured to mate with the connector region 135. In some embodiments, the internal portion of the surgical tool can also be in the shape of the external surface of the introducer cone 130, as shown in FIG. 9B. This configuration will allow the surgical tool to mate well with the insertion end 180 of the modular rod 110.

### EXAMPLE

FIG. 1 shows an example of a THA procedure. A main incision 105 is shown that may create a 45/45/90 degree triangle from the portal incision 101 to the main incision 105 to the acetabulum 115. The guide wire 120 is inserted through the portal incision 101 and through the tissue until it reaches the acetabulum 115. Because the guide wire is thin, very little tissue damage occurs as a path is created to the acetabulum. With the guide wire 120 in place, now a path for the modular rod 110 is established. The exposed portion of the guide wire 120 is inserted into the channel 125 of the modular rod 110 and the modular rod 110 is advanced towards the acetabulum 115. Preferably, the introducer cone 130 and one or more connector regions 135 create a seamless, smooth structure that passes through soft tissue with minimal trauma to the tissue following the guide wire 120 to the face of the acetabulum 115. Once the introducer cone 130 and connector region 135 is positioned in the acetabulum 115 so as to be accessible through the main incision 105, the surgeon may remove the guide wire 120 by pulling it out through the internal channel 125 of the modular rod 110. Additionally, the surgeon may then place any one of a number of end attachments onto the introducer cone 130 and connector region 135 to prepare the acetabulum 115 and then to place and orient a new acetabular cup 140, or perform some other aspect of the THA.

Once properly in place, the access end 185 of the modular rod 110 may protrude out of the body of the patient at the portal incision 101. The introducer cone 130 and connector region 135 end protrudes through the portal incision 101 path towards the acetabulum 115. An acetabular component or some other tool may be positioned near the acetabulum 115 through the main incision 105 to receive the connector region 135 and introducer cone 130. There, the introducer cone 130 and connector region 135 can be connected, at various appropriate times during the procedure, to a series of surgical tools depending on the stage of the surgery. For example, a first surgical tool can be attached to the connector region 135 and introducer cone 130 and its function performed. When complete, the first tool is detached from connector region 134 and introducer cone 130, using any appropriate tool necessary, and the first surgical tool is removed through the main incision 105. Then, a second surgical tool can be inserted through the main incision 105 and attached to the connector region 135 and introducer cone 130 to perform a second function. Advantageously, the modular rod 110 need not be removed from the subject in order to attach the second surgical tool. Once the second function is complete, the second surgical tool can be detached from the connector region 135 and the introducer cone 130 and removed from the subject through the main incision 105. A third surgical tool, as necessary, can be inserted through the main incision 105 and attached to the connector region 135 and introducer cone 132 perform a third function. When the third function is complete, the third surgical tool can be removed without having to remove the modular rod 110. This process can repeat as many times as necessary with subsequent surgical tools to advance the surgical procedure.

As stated above, in some embodiments the modular rod 110 may also use surgical tools that articulate (i.e., form a joint), such as shown in FIG. 8, to create greater precision in the surgery. Alternatively, an articulating structure could be built into the introducer cone 130 and connector region 135 of the rod itself to allow the surgical tool to articulate relative to the modular rod 110.

Another example of use of the present invention would be the surgeon coupling the modular rod 110 shaft with a removal blade 150, such as the one shown in FIG. 5, and then applying a force to the access end of the shaft, pushing the shaft longitudinally downwards and driving the removal blade 150 into a portion of an existing acetabular component 140. The old acetabular component 140 may often be fixed or at least partially fixed in the acetabulum 115 of the pelvis due to promoted bone growth or the like. The surgeon may then apply a rotary force to the access end of the same shaft, through the portal incision 101 path rotating the blade to thereby dislodge and remove the old acetabular component 140. A central post for center stabilization, which allows blades of appropriate diameter to be used to precisely carve a space between an acetabular component 140 that may be adhered to the overlying bone may be used as a pivot point, possibly with a concentric ball or an attachment directly to the center hole of the old acetabular component 140.

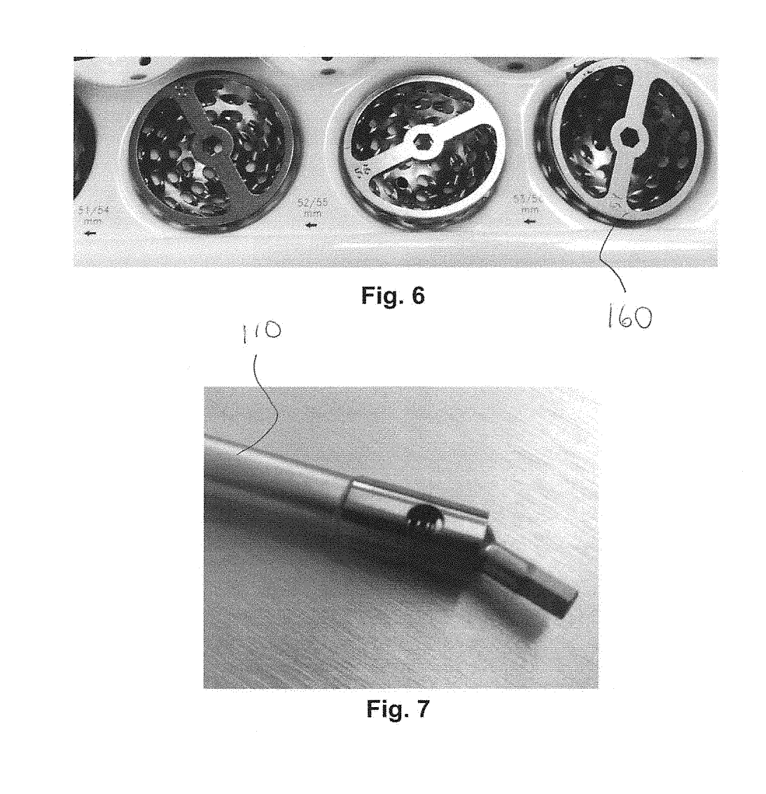

The surgeon may then decouple the removable blade 150, and remove the blade and the original acetabular component 140 through the main incision 105. Without almost any movement of the shaft, the surgeon may insert a new surgical tool, such as a reamer 160, cutting tool, impactor 170, grinder 155, burr 195, sander, or the like, such as the grinder 155 end shown in FIGS. 4A and 4B or the selection of reamers 160 shown in FIG. 6, through the main incision 105 and position it near the acetabulum 115 for coupling with the connector 135 end of the shaft.

The new tool may be moved into place inside the acetabulum 115, and powered as needed by battery, gas, etc. to for example, prepare the acetabulum 115 to receive a new acetabular component 140. The surgeon may then decouple the new tool from the modular rod 110, remove the new tool from the acetabulum 115 through the main incision 105, and continue this process with any series of tools until the acetabulum 115 is ready to receive a cup placement device 165.

Additionally, the surgeon may reinsert the guide wire 120 and remove the modular rod 110 to replace the modular rod 110 with a modular rod 110 of a differing strength or functional purpose throughout the procedure. Various embodiments of the modular rod 110 may be used for situations where there may be a need for a modular rod 110 with the strength necessary for transferring a longitudinal impact force along its length, or for a strength necessary to transfer a rotary force along its length. Once the acetabulum is prepared, the surgeon may insert the cup placement device 165 through the main incision 105, and may couple the modular rod 110 to the cup placement device 165 (such as shown in FIG. 8), place the new acetabular component 140 in position, decouple the cup placement device 165, and couple the impactor 170 (such as shown in FIG. 9B). The surgeon may impact the acetabular component 140 while the cup placement device 165 is attached or after it is removed to fine tune its placement and orientation and ultimately seat the acetabular component 140 with or without the use of an adhesive.

Adjustable Clamp

In some embodiments, to stabilize the modular rod 110, an adjustable clamp 190 may be applied to the modular rod 110. The adjustable clamp 190 may be with or without a handle. The adjustable clamp 190 may be adjustable along the length of the modular rod 110 and serves to stabilize the modular rod 110 in place at the surface of the skin. Therefore, when the modular rod 110 is in a desired position within the subject, the adjustable clamp 190 may be slid down along the rod until the adjustable clamp abuts against the subject's skin. The adjustable clamp 190 may be secured to the modular rod 110 and the subject to keep the modular rod 110 in place.

In some embodiments, the adjustable clamp 190 may be a flat device with the hole therethrough. Preferably, the adjustable clamp 190 is a ring-shaped device. The modular rod 110 can be inserted into the hole of the adjustable clamp 190 and the adjustable clamp can be positioned at the proper location along the modular rod 110 to place the modular rod 110 at the desired position. The hole of the adjustable clamp 190 is configured to slide along the modular rod 110 and be fixed at the desired position on the modular rod 110.

Some embodiments may include an adjustable clamp 190 having a cushioned ring 145 to protect the skin while providing a firm stop. This stabilizing function is important in reducing unwanted excursion of the modular rod 110, further minimizing risk of soft tissue trauma. Thus, the adjustable clamp 190 may prevent the modular rod 110 from moving too far into the hip joint when it is not being used, but the adjustable clamp 190 may readily be removed or advanced up the modular rod 110 towards the access end to allow the modular rod 110 to be introduced further when required.

As described above, some embodiments may also include a cushioned ring 145, such as the one shown in FIG. 10 that can be applied to the modular rod 110. This cushioned ring 145 may be larger than the opening in the joint capsule created by the modular rod 110. The cushioned ring 145 may be 15-20 mm in diameter and approximately 1-5 mm thick. The cushioned ring 145 may also have a slight concavity or a large concave radius of curvature to allow further blending with the adjacent soft tissues. The cushioned ring 145 may function to further stabilize the modular rod 110 and limit its ability to be drawn upward into the soft tissue channel.

Removable Segments

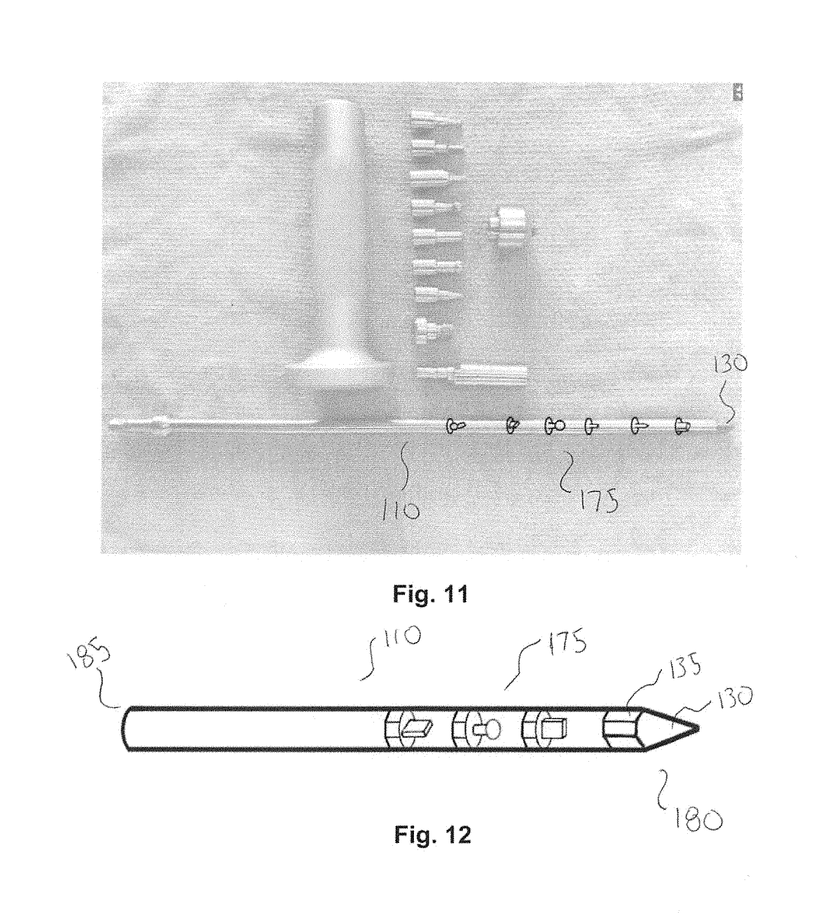

In another embodiment as shown in FIG. 12, the modular rod 110 may have an end portion 175 at the insertion end 180 that is approximately 1-inch to approximately 5-inches long, or longer. The end portion 175 may comprise a series of removable pods, which are individual removable segments of surgical tools in a stacked configuration. The pods can be organized before any surgery in a desired sequence. For example, the pod at the most distal end of the modular rod 110 can have the first surgical tool needed in the surgical process. The second pod, moving away from the insertion end 180 and towards the access end 185, can have the second surgical tool needed in the surgical process. The third pod adjacent to the second pod and moving towards the access end 185 can have a third surgical tool needed in the third step of the surgical process. This continues on for as long as the modular rod 110 can handle.

In use, the surgeon uses the first pod in the surgical process. When complete, the surgeon removes the first pod by detaching it from the second pod and removing it from the subject through the main incision 105. The second pod is now exposed and ready for use. The surgeon can then perform the second step of the surgical procedure using the second pod. When complete, the second pod is detached from the third pod and removed from the subject through the main incision 105. Now, the third pod is exposed and ready for use. This process continues on until the surgical procedure is complete. If more pods are required to complete the surgical procedure than the modular rod 110 can hold, additional modular rods 110 with the necessary pods can be used.

The pods can be attached to each other with any type of fastening mechanisms described herein. Preferably, the pods are comprised of their own connection region 135 and a surgical tool.

For example, when the modular rod 110 is initially introduced, the first pod at the end portion 175 can have a removable segment that has the introducer cone 130 and connection region 135 configuration described above. By virtue of its sharp, or at least graduated, tip, the introducer cone 130 may help create the channel from the portal incision 101 to the hip joint to assist in penetrating through the tissue channel into the hip cavity.

The introducer cone 130 itself may be faceted or otherwise configured to function as a powered rotational driver for various attachable tools. These attachable tools may be used for cutting (to remove or penetrate hard bone) or for shaving (such as an acetabular reamer 160), or as a power screwdriver (see, FIG. 11, second tip from top, for placing or removing threaded devices, such as screws or adapters). These attachable tools may be attached to the introducer cone 130 or to the connection region 135 adjacent to the introducer cone 130 using the main incision to gain access to the introducer cone 130 and connection region 135.

Alternatively, the introducer cone 130 may be removed exposing the pod. The next pod may have a multi-axis screwdriver that can articulate relative to the longitudinal axis of the modular rod 110—in the event that a screw, adaptor, or the like might need to be screwed into a position that is off this main axis of the modular rod 110. The screwdriver could be “deployed”, exposed, or activated, by removing the original introducer cone 130. Thus, with this configuration, the surgeon is not required to mount the screwdriver tip within the small cavity and through the tight access of the main incision 105 to use the modular rod 110 as a screwdriver, but rather, the screwdriver tip is already resident within the pod or end portion 175 of the rod. All the surgeon has to do is to gently remove the introducer cone 130 or any other preceding segment to expose or deploy the screwdriver tip. This single step of removal has shown to be a much easier step to perform than attempting to mount such a tip at the end of the modular rod 110 while the modular rod 110 remains resident in the patient and accessible through the main incision 105. This process can continue to expose the next surgical tool, and so on.

In such an embodiment, various tips may be provided, resident within the modular rod 110, and they may be provided in the sequence that is most commonly needed for a given procedure. That is, the modular rod 110 may provide a series of modular tips to be used in a functional sequence for a given surgical procedure. In fact, the surgeon himself or herself may assemble the modular rod 110 in the order of the expected sequence of steps for the given procedure and the given needs of the patient.

The surgeon may first use the introducer cone 130 at the beginning to help pass through the skin and tissues of the patient while avoiding the trauma of cutting particular sensitive tissues to establish a corridor for the modular rod 110. Then, for a second step of the procedure, the surgeon may wish to mount a cutting device to the introducer cone 130 for cutting away an old implant. After that, the surgeon may remove the cutting device and the introducer cone 130 to expose the next segment, such as an articulating multi-axis screwdriver to perform a third step, such as removing an old implant. Once this third surgical step is performed, this segment and tool may be removed to expose and deploy a third segment configured to assist the surgeon in performing a third step of the procedure. This third step may be the need to shave or file the surfaces of the acetabulum 115 to ready it to receive a new prosthesis. Again, the modular rod 110 itself, with the chosen segment deployed, may power or drive this shaving motion.

Once the acetabulum 115 is ready to receive the new prosthesis, the buffer or other shaving attachment may be removed, exposing a mounting segment for assisting the surgeon in mounting and positioning the new acetabular component 140. Once the acetabular component 140 is roughly in position, the mounting segment may be removed, exposing and deploying the next chosen segment, such as an impaction tool 170 designed to transfer and impart a force on an acetabular liner or the acetabular component 140 itself sufficient to alter the orientation of the acetabular component 140 so that the surgeon may move the prosthesis into the precise orientation desired. Once this step is completed, the surgeon may remove this segment, exposing yet further segments if desired. One potential embodiment of a modular rod 110 with segmented and built in tools is shown in FIG. 12.

In one embodiment as shown in FIG. 12 the pod or individual segments of the end portion 175 may be press fit upon on the end of the next successive segment and on the end portion 175 of the multi-function, modular rod 110 itself. Or, the connections could be magnetic or threaded (clockwise or counterclockwise, depending on the rotational force to be applied), or could be one of the connections discussed above. FIG. 11 shows another potential embodiment of a modular rod 110 and a plurality of existing attachments that may be implemented into the individual segments of the modular rod 110 for incrementally detaching during a surgery.

While the present invention has been described with regards to particular embodiments, it is recognized that additional variations of the present invention may be devised without departing from the inventive concept.

What is claimed is:

1. A device for arthroplasty procedures, comprising:
a. a modular rod, having an attachment end and a insertion end;
b. a channel, extending along a length of the modular rod through the attachment end and the insertion end;
c. a guide wire insertable through the channel;
d. a connector region on the attachment end;
e. an introducer cone adjacent to the connector region;
f. an attachment device having a receiving connector for accepting the introducer cone and the connector region;
g. wherein the guide wire is configured to be inserted into a subject;
h. wherein the modular rod is configured to be guided into the subject along the guide wire;
i. wherein the connector is configured to allow removal of the attachment device while remaining within the subject;
j. wherein the connector is configured to allow for attachment of a subsequent attachment device while remaining within the subject;
k. wherein the introducer cone has all smooth edges;
l. wherein the guide wire has a diameter of less than 5 mm and rigid enough to pass through tissue without bending.

2. The device for arthroplasty procedures of claim 1, wherein the channel is an external and off center channel for receiving the guide wire.

3. The device for arthroplasty procedures of claim 1, wherein the connector region is selected from the group consisting of: threading, keyed, socket, detent, catch, clip, magnet, hook, chunk, and clamp.

4. The device for arthroplasty procedures of claim 1, wherein the modular rod is configured to utilize attachments that articulate and form a joint.

5. The device for arthroplasty procedures of claim 1, wherein the modular rod is configured to accept an adjustable clamp to stabilize the modular rod in place at a surface of the subject's skin.

6. A device for arthroplasty procedures, comprising:
a. a modular rod, having an attachment end and a insertion end;
b. a channel, extending through a length of the modular rod through the attachment end and the insertion end;
c. a guide wire insertable through the channel;
d. a connector region on the attachment end;
e. an introducer cone adjacent to the connector region; and
f. an attachment device, having a receiving connector for accepting the introducer cone and the connector region.

7. The device for arthroplasty procedures of claim 6, wherein the channel is an external and off center channel for receiving the guide wire.

8. The device for arthroplasty procedures of claim 6, wherein the connector region is selected from the group consisting of: threading, keyed, socket, detent, catch, clip, magnet, hook, chunk, and clamp.

9. The device for arthroplasty procedures of claim 6, wherein the guide wire has a diameter of less than 5 mm and rigid enough to pass through tissue without bending.

10. The device for arthroplasty procedures of claim 6, wherein the introducer cone has only smooth edges.

11. The device for arthroplasty procedures of claim 6, wherein the modular rod is configured to utilize attachments that articulate and form a joint.

12. The device for arthroplasty procedures of claim 6, wherein the modular rod is configured to accept an adjustable clamp to stabilize the modular rod in place at a surface of the human's skin.

13. A method for conducting arthroplasty procedure, comprising:
a. creating a main incision and a portal incision in a subject;
b. mapping out a path from the portal incision to an acetabulum for a guide wire using radiography techniques;
c. inserting the guide wire along the path from the portal incision to the acetabulum;
d. using the guide wire to guide a modular rod configured with an introducer cone and a connector region at an insertion end of the modular rod toward the acetabulum until the introducer cone and the connector region are accessible through the main incision;
e. removing the guide wire from the subject;
f. inserting an acetabular component through the main incision and positioning the acetabular component near the acetabulum to receive the modular rod.

14. The method for conducting arthroplasty procedures of claim 13, wherein the acetabular component is removed through the main incision and a series of surgical tools are subsequently inserted and removed throughout the arthroplasty procedure.

15. The method for conducting arthroplasty procedures of claim 13, further comprising attaching an attachment that articulates and forms a joint to the modular rod.

16. The method for conducting arthroplasty procedures of claim 13, further comprising inserting the guide wire back through the modular rod, removing the modular rod, and replacing with a second modular rod of a differing functional purpose.

17. The method for conducting arthroplasty procedures of claim 13, further comprising stabilizing the modular rod in place at a surface of the subject's skin with an adjustable clamp.

18. The method for conducting arthroplasty procedures of claim 13, further comprising removing one or more removable pods from the insertion end.

19. The method for conducting arthroplasty procedures of claim 18, wherein the removable pods are various tools used in arthroplasty procedures.
